(19) United States
(12) Patent Application Publication
(10) Pub. No.: US 2012/0130150 A1
(43) Pub. Date: May 24, 2012

(54) APPARATUS AND METHOD OF TREATMENT UTILIZING A VARYING ELECTROMAGNETIC ENERGIZATION PROFILE

(75) Inventor: Jeffrey D. Edwards, Leederville (AU)

(73) Assignee: International Scientific Pty Ltd.

(21) Appl. No.: 13/378,971

(22) PCT Filed: Jun. 22, 2010
(86) PCT No.: PCT/AU10/00782

§ 371 Date: Jan. 24, 2012

(30) Foreign Application Priority Data
Jun. 24, 2009 (AU) 2009902910

(51) Int. Cl. A61N 2/04; A61N 2/00

(52) U.S. Cl. 600/14; 600/9

(57) ABSTRACT

The present invention relates to an apparatus and method for improving the delivery of desirable substances across a membrane utilizing a varying electromagnetic energisation profile. More particularly, the invention provides methods for modifying the cellular environment of such membranes and the behaviour of substance molecules during transport across a membrane. In use, the profile will have application in, enhancing transmembrane delivery of substances (such as pharmaceuticals, nutraceuticals, biopharmaceuticals and cosmeceuticals), and/or enhancing or expanding a cellular cohort of membranes and surrounding tissues.

### FIELD OF THE INVENTION

The present invention relates to an apparatus and method of improving the delivery of a compound across a membrane utilizing a varying electromagnetic energisation profile. More particularly, the invention provides methods for modifying the cellular environment of such membranes and the behavior of molecules during transport across a membrane. In use, the profile will have application in, enhancing transmembrane delivery of substances (such as pharmaceuticals, nutraceuticals, biopharmaceuticals and cosmeceutical), and/or enhancing or expanding a cellular cohort of membranes and surrounding tissues.

### BACKGROUND OF THE INVENTION

The cornerstone of modern human and animal treatments is to be able to treat an individual quickly and efficiently without physical intervention. Such treatments are not limited to medicinal applications but also include cosmetic and structural improvements which have the capacity to alter an individual's physical form including their appearance. There are a myriad of different treatments available for achieving such end results.

In the medical field, treatment (be it prophylactic or therapeutic) involves the delivery of substances such therapeutic substances or cosmeceutical to a desired treatment area, the substances must be physically and/or chemically available to the treatment area, and must be available in a sufficient concentration to exert a beneficial biological effect.

As an alternative to conventional methods of delivery, transmembrane delivery techniques have been developed so that a degree of site specificity is obtained and a desired concentration of substance is achieved which is unaltered by digestion or blood chemistry. Transmembrane delivery techniques also offer the possibility of high user compliance, ease of management, low toxicity and high cost effectiveness.

A membrane that poses a significant barrier to entry for many therapeutic substances is the skin because the lipid bilayer of the stratum corneum skin layer generally only allows very small neutrally charged particles of the order of 1 nm to pass through. As such, transdermal delivery of many ions, drugs, macro molecules, DNA fragments, genes and therapeutic substances is problematic.

In one transdermal technique referred to as iontophoresis, an electrical energy gradient is used to mobilize a target molecule and an electrical voltage is employed to accelerate the charged target molecule between conductors of opposing potentials, the electrical gradient being sufficient to cause the movement of target molecule through a membrane.

However, such methodology is only applicable to certain charged molecules and requires an electrically conductive medium to be maintained in contact with the membrane. In addition, the introduction of charged ions by such means can result in the displacement of a beneficial ion of the opposite charge leading to limitations of use and potentially undesirable effects.

A further transdermal delivery technique is referred to as electroporation. With this technique, successive pulses of electrical current of 1 ms to 10 ms duration of the order of 100 to 200 volts are directly applied to a target skin area or membrane using electrically conductive probes or electrodes. Such charge disrupts the orderly arrangement of components that make up dermal, cellular and other membranes leading to the formation of pores or holes through which molecules may pass.

However, as with the iontophoresis technique, since relatively high energy levels are used may cause cellular damage, discomfort to the user and may result in damage to the membrane that requires extensive period to recover. In addition, in view of the high voltages employed, electroporation is of limited use many in vivo applications.

The barrier effect of the stratum corneum arises as a result of the intercellular lipid matrix which comprises long chain ceramides, free fatty acids, cholesterol and other lipids. The lipids are arranged into bilayers having hydrocarbon chains aligned to form an oily bilayer core and electrically charged or polar outwardly facing head groups. This produces a highly selective filter-like structure. In contrast to phospholipid bilayer membranes found elsewhere in the body, the composition of the stratum corneum lipid bilayers is a much more rigid and ordered structure. As a consequence, the barrier to penetration of the stratum corneum by therapeutic substances is much greater compared to the corresponding barriers to penetration produced by other body membranes.

Therapeutic substance delivery techniques such as iontophoresis and electroporation rely on energy to disrupt the stratum corneum lipid bilayer, which disrupts the hydrophilic-hydrophobic orientation of the bilayer and creates regions of random orientation or pores through which some substances may be introduced.

There exists a strong demand for a new method for modifying a cellular environment across a membrane, which ameliorates one or more of the above identified problems.

### SUMMARY OF THE INVENTION

In accordance with an aspect of the present invention, there is provided an apparatus for modifying a cellular environment across a membrane, said apparatus comprising:
means for producing and delivering an electromagnetic field to the cellular environment wherein the field delivered is defined by a mnemonic profile of:

[(A-B-C-D)E],[A₁-B₁-C₁-D₁)E₁]
where,
A and A₁ respectively define the number of 400 μs time units that the electromagnetic field pulse is on for wherein each of A and A₁ is a number between 0.1 and 10;
B and B₁ respectively define is the number of 400 μs time units the field is off for wherein B is a number between 0.1 and 100, while B₁ is a number between 0.1 and 100;
C is a number between 1 and 255, which defines the number of times the A and B combination is repeated, while C₁ is also a number between 1 and 255 and defines the number of times the A₁ and B₁ combination is repeated;
D and D₁ respectively define the number of 400 μs time units that the field is off for wherein D is a number between 0 and 255, while D₁ is a number between 0 and 255; and
E defines the number of times the A to D envelope is executed before moving onto the [(A₁-B₁-C₁-D₁)E₁] packet, while E₁ defines the number of times the A₁ to D₁ envelope is executed before moving onto the next mnemonic profile wherein E and E1 are respectively numbers between 1 and 25; and
wherein during use when the electromagnetic field is incident on a patient, the cellular environment across the membrane is modified.

In use, the profile will have application in, enhancing transmembrane delivery of substances (such as pharmaceuticals, nutraceuticals, biopharmaceuticals and cosmeceutical), cellular cohort of membranes and surrounding tissues.

In accordance with an alternative aspect of the present invention, there is provided a method of transmembrane delivering of substances (such as pharmaceuticals, nutraceuticals, biopharmaceuticals and cosmeceutical), said method comprising:
producing an electromagnetic field defined by a mnemonic profile of:

[(A-B-C-D)E],[(A₁-B₁-C₁-D₁)E₁]
where,
C is a number between 1 and 255, which defines the number of times the A and B combination is repeated, while C₁ is also a number between 1 and 255 and defines the number of times the A₁ and B₁ combination is repeated;
D and D₁ respectively define the number of 400 μs time units that the field is off for wherein D is a number between 0 and 255, while D₁ is a number between 0 and 255; and
E defines the number of times the A to D envelope is executed before moving onto the [(A₁-B₁-C₁-D₁)E₁] packet, while E₁ defines the number of times the A₁ to D₁ envelope is executed before moving onto the next mnemonic profile wherein E and E1 are respectively numbers between 1 and 25; and
directing the electromagnetic field at a desired treatment area of a patient.

Other aspects and advantages of the invention will become apparent to those skilled in the art from a review of the ensuing description, which proceeds with reference to the following illustrative drawings.

### BRIEF DESCRIPTION OF THE DRAWINGS

FIG. 1 is a diagrammatic perspective view of a portion of a stratum corneum prior to application of an electromagnetic field produced in accordance with an apparatus and method according to the present invention;

FIG. 2 is a diagrammatic perspective view of the stratum corneum shown in FIG. 1 during application of an electromagnetic field produced by an apparatus and method in accordance with an embodiment of the present invention;

FIG. 3 is a schematic diagram of an energisation signal used to effect energisation of an electromagnetic field generation device of an apparatus in accordance with an embodiment of the present invention;
A and A₁ respectively define the number of 400 μs time units that the electromagnetic field pulse is on for wherein each of A and A₁ is a number between 0.1 and 10;
B and B₁ respectively define is the number of 400 μs time units the field is off for wherein B is a number between 0.1 and 100, while B₁ is a number between 0.1 and 100;
C is a number between 1 and 255, which defines the number of times the A and B combination is repeated, while C₁ is also a number between 1 and 255 and defines the number of times the A₁ and B₁ combination is repeated;
D and D₁ respectively define the number of 400 μs time units that the field is off for wherein D is a number between 0 and 255, while D₁ is a number between 0 and 255; and
E defines the number of times the A to D envelope is executed before moving onto the [(A₁-B₁-C₁-D₁)E₁] packet, while E₁ defines the number of times the A₁ to D₁ envelope is executed before moving onto the next mnemonic profile wherein E and E1 are respectively numbers between 1 and 25; and
directing the electromagnetic field at a desired treatment area in or on a patient, which area is also exposed to the therapeutic substance.
   In accordance with yet another alternative aspect of the present invention, there is provided a method of enhancing or expanding a cellular cohort in or on a patient said method comprising:
producing an electromagnetic field defined by a mnemonic profile of:

[(A-B-C-D)E],[(A₁-B₁-C₁-D₁)E₁]
where,
A and A₁ respectively define the number of 400 μs time units that the electromagnetic field pulse is on for wherein each of A and A₁ is a number between 0.1 and 10;
B and B₁ respectively define is the number of 400 μs time units the field is off for wherein B is a number between 0.1 and 100, while B₁ is a number between 0.1 and 100;

FIG. 4 is an enlarged schematic diagram of an energisation signal packet of the energisation signal shown in FIG. 3;

FIG. 5 is a schematic diagram illustrating circuitry of an apparatus for facilitating transdermal delivery of therapeutic substances in accordance with an embodiment of the present invention; and

FIG. 6 is a diagrammatic perspective view of an apparatus for facilitating transdermal delivery of therapeutic substances in accordance with an embodiment of the present invention, the apparatus including the circuitry shown in FIG. 5.

FIG. 7 represents the averaged cumulative amount of diclofenac (Voltaren®) in the receptor chamber of a standard Franz-type diffusion set-up for either passive or Dermaportation induced penetration through excised human epidermis. Dermaportation was switched on from 30-60 min only (grey bar).

FIG. 8 represents the average cumulative amount of hydrocortisone in the receptor chamber of a standard Franz-type diffusion set-up is depicted for passive and Dermaportation induced penetration through excised human epidermis. Dermaportation was switched on from 0-240 min (grey bar).

FIG. 9 represents the immune response of sheep to Dermaportation-enhanced topical vaccination is similar to the immune response to intramuscular vaccination. Blood samples were taken before vaccination, 2 weeks after, and 2 weeks post booster vaccination.

FIG. 10 represents the effects of passive or Dermaportation induced transdermal delivery of tetracaine (Ametop®) on touch sensitivity thresholds measured via an electronic von Frey system, before, immediately after, and 20 min post topical administration. The topical administration time was 20 min.

FIG. 11 represents the cumulative amount of ALA penetrating human epidermis following application of 2% ALA solution with Dermaportation (0-4 h) or passive diffusion: mean±sem, n=4/3

FIG. 12 represents Cumulative amount of ALA penetrating human epidermis following application of 20% ALA solution with Dermaportation (0-4 h) or passive diffusion: mean, n=2.

FIG. 13 represents the average cumulative amount of naltrexone in the receptor chamber of a standard Franz-type diffusion set-up is depicted for passive and Dermaportation induced penetration through excised human epidermis. Dermaportation was switched on from 0-240 min (grey bar)

### DESCRIPTION OF AN EMBODIMENT OF THE INVENTION

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values. A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

### Embodiments

The inventors of the present invention have discovered that a cellular environment can be advantageously manipulated when exposed to a unique electromagnetic mnemonic profile that induces molecular activation of molecules at an atomic level. When such molecules are associated or bound by atomic bonds, they are susceptible to electromagnetic influences and are subject to diffusion rather than Brownian motion. Exposing such molecules to a magnetic field induces a tendency for organized alignment in the molecules, with the kinetic energy component of the molecular moment being converted into diamagnetic repulsion in a direction away from the magnetic field source. In a cellular environment, this can either be used to direct the passage of substances through that membrane into the environment or can be used to enhance pathways through which substance molecules pass by altering their relationship with loosely or tightly bound water.

Advantages associated with such drug delivery include, without limitation: (a) that needles and the associated pain are avoided; (b) that patient compliance of drug regimens is significantly improved; (c) that the apparatus and method offer prolonged or sustained delivery, potentially over several days to weeks. Other delivery methods, such as oral or pulmonary delivery, typically require that the drug be given repeatedly to sustain the proper concentration of drug within the body. With sustained delivery according to the invention, dose maintenance is performed automatically over a long period of time. This is especially beneficial for drugs with short half-lives in the body, such as peptides or proteins. Also drug molecules that only have to cross the skin to reach the bloodstream when given transdermally can bypass first-pass metabolism in the liver, and also avoid other degradation pathways such as the low pH's and enzymes present in the gastrointestinal tract.

Therefore, in accordance with an aspect of the present invention, there is provided an apparatus for modifying a cellular environment across a membrane, said apparatus comprising:
means for producing and delivering an electromagnetic field to the cellular environment wherein the field delivered is defined by a mnemonic profile of:

[(A-B-C-D)E],[(A₁-B₁-C₁-D₁)E₁]
where,
A and A₁ respectively define the number of 400 μs time units that the electromagnetic field pulse is on for wherein each of A and A₁ is a number between 0.1 and 10;
B and B₁ respectively define is the number of 400 μs time units the field is off for wherein B is a number between 0.1 and 100, while B₁ is a number between 0.1 and 100;
C is a number between 1 and 255, which defines the number of times the A and B combination is repeated, while C₁ is also a number between 1 and 255 and defines the number of times the A₁ and B₁ combination is repeated;
D and D₁ respectively define the number of 400 μs time units that the field is off for wherein D is a number between 0 and 255, while D₁ is a number between 0 and 255;
E defines the number of times the A to D envelope is executed before moving onto the [(A₁-B₁-C₁-D₁)E₁] packet, while E₁ defines the number of times the A₁ to D₁ envelope is executed before moving onto the next mnemonic profile wherein E and E1 are respectively numbers between 1 and 25; and
wherein during use when the electromagnetic field is incident on a patient, the cellular environment across the membrane is modified.

As used herein a “cellular environment” is directed to, but not specifically limited to, the external and/or internal factors, compounds or conditions that are involved in influencing, inducing, effecting or stimulating either cells or tissue within the proximity of a treatment area.

As used herein “membrane” is directed to a (a) thin flexible sheet of tissue connecting, covering, lining, or separating various parts or organs in an animal's body, or forming the external wall of a cell therein, (b) a thin, pliable, and often porous sheet of any natural or artificial material, (c) a casing, covering, exterior surface or a cellular sleeve, or (d) an epidermal or dermal layer covering an animal.

According to the invention the mnemonic profile selected will differ according the drug administered. The following table illustrates various field parameters for a range of different drugs.

| Field parameters | Compound |
|---|---|
| [(1, 1, 12, 255)5], [(1, 35, 72 ,0)2] | Diclofenac |
| | diethylammonium salt |
| [(1, 1, 12, 255)5], [(1, 35, 72, 0)2] | Hydrocortisone in PBS |
| [(1, 1, 12, 255)5], [(1, 35, 72, 0)2] | GLANVAC vaccine |
| [(1, 1, 24, 255)5], [(1, 35, 72, 0)2] | Tetracaine HCL |
| [(1, 1, 12, 255)5], [(1, 35, 72, 0)2] | Tetracaine (4%) |
| [(1, 1, 12, 255)5], [(1, 35, 72, 0)2] | Ametop (tetracaine gel) |
| [(1, 1, 12, 255)5], [(1, 35, 72, 0)2] | 5-aminolevulinic acid |
| [(1, 1, 12, 255)5], [(1, 35, 72, 0)2] | Naltrexone in PBS |
| [(1, 1, 12, 255)5], [(1, 35, 72, 0)2] | 5 mg/ml Lidocaine HCL |
| | in PBS |
| [(1, 1, 12, 255)5], [(1, 35, 72, 0)2] | 5 mg/ml Lidocaine HCL |
| | in PBS |
| [(1, 1, 12, 255)5], [(1, 35, 72, 0)2] | Lidocaine in PBS |
| [(1, 1, 12, 255)5], [(1, 35, 72, 0)2] | Lidocaine HCL, |
| | prilocaine HCL |
| | combination,, PBS |
| [(1, 1, 12, 255)5], [(1, 35, 72, 0)2] | Lidocaine in PBS |
| [(1, 1, 255, 0)1], [(1, 1, 255, 0)1] | |
| [(1, 1, 12, 255)5], [(1, 35, 72, 0)2] | 5 mg/ml Lidocaine HCL |
| | in PBS |
| [(1, 1, 12, 255)5], [1, 35, 72, 0)1] | |
| [(1, 1, 12, 255)5], [(1, 35, 72, 0)2] | 5 mg/ml Lidocaine HCL |
| | in PBS |
| [(1, 1, 12, 255)10], [(1, 35, 72, 0)2] | |
| [(1, 1, 12, 255)5], [(1, 35, 72, 0)2)] | 5 mg/ml Lidocaine HCL |
| | in PBS |
| [(1, 1, 24, 255)5], [(1, 35, 72, 0)2] | |
| [(1, 1, 12, 255)5], [(1, 35, 72, 0)2] | Lidocaine HCL |
| [(1, 1, 24, 255)5], [(1, 35, 72, 0)2] | |
| [(1, 1, 24, 255)5], [(1, 35, 72, 0)2] | Lidocaine Hcl 5 mg/ml |
| [(1, 1, 12, 255)5], [(1, 35, 72, 0)2] | Lidocaine HCL 5 mg/ml |
| [(1, 1, 2, 255)5], [(1, 1, 2, 255)5] | |
| [(1, 1, 12, 255)5], [(1, 35, 72, 0)2] | Lidocaine HCL 5 mg/ml |
| [(2.5, 255, 3), 255)10], [(2.5, 255, 3), 255)10] | |
| [(1,1, 12, 255)5], [(1, 35, 72, 0)2] | Lidocaine HCL 5 mg/ml |
| [(2.5, 255, 3), 255)10], [(2.5, 255, 3), 255)10] | |
| [(1, 1, 12, 255)5], [(1, 35, 72, 0)2] | Lidocaine HCL 5 mg/ml |
| [(2.5, 2.5, 2)25000)5], [(−2.5, −2.5, 2)25000)1] | |
| [(1,1, 12, 255)5], [(1, 35, 72, 0)2] | Lidocaine HCL 5 mg/ml |
| [(2.5, 2.5, 2)25000)5], [(−2.5, −2.5, 2)25000)1] | |

The apparatus of the invention will have a wide variety of applications where application in, enhancing transmembrane delivery of substances, and/or enhancing or expanding a cellular cohort.

As used herein a “substance” is directed to pharmaceuticals, nutraceuticals, biopharmaceuticals, cosmeceuticals or any other substance desired to be passed through a membrane to a patient for the purpose of obtaining a beneficial effect. It will be appreciated that such substances may be either therapeutic of prophylactic in nature. For example, it may be a drug, vaccine, ion, phytochemicals, enzymes, antioxidants, herbs, spices, natural or semi-natural or refined plant extracts, oils, essential oils, vitamin, nutrient, macromolecule, DNA fragment, gene, protein, amino acid sequence or any other substance desired to be passed through a membrane of a patient for the purpose of obtaining a beneficial effect. More preferably, the substance is a product that is well-researched and tested for mildness, efficacy, biodegradability, low toxicity, cleansing ability, emulsification, moisturisation, skin appearance and feel, smell (fragrance) and lubrication.

Substances suitable for use in the invention may be in the form of liquids, solutions, suspensions, emulsions, solids, semi-solids, gels, foams, pastes, ointments, or triturates. They may also be mixed with a range of excipients including penetration enhancers, adhesives and solvents.

To the extent that the apparatus of the invention has uses in enhancing or expanding a cellular cohort, the apparatus is applied to the tissue or membrane in or on which the cellular expansion is to be achieved. For example the apparatus might be applied to skin to expand a corneocyte or keratinocyte population.

In one arrangement, the means for producing an electromagnetic field includes a capacitively coupled plate or coil. The means for producing an electromagnetic field may further include a solid state switching device which may be a transistor such as a bipolar transistor connected in series with the coil.

In one arrangement, the apparatus includes a control means arranged to produce an energisation signal useable to control switching of the solid state switching device, the energisation signal including a repeating energisation signal packet, each energisation signal packet including a plurality of energisation signal pulses of generally rectangular configuration.

The control means may comprise a microcontroller which may be programmable by a user. The microcontroller may be programmed such that during drug delivery permeability across the membrane is increased at one or more specific times, permeability is increased for a specific period of time, and so on.

In one embodiment, the energisation of each electromagnetic pulse is at a frequency of between 1 Hz and 100 Hz, more particularly between 10 Hz and 50 Hz.

In one arrangement, the substance is disposed on a surface of the apparatus. The substance may be a pharmaceutical, nutraceutical, biopharmaceutical and cosmeceutical or any other substance desired to be passed through the membrane of a patient for the purpose of obtaining a beneficial effect.

In accordance with an alternative aspect of the present invention, there is provided a method of transmembrane delivery of substances (such as pharmaceuticals, nutraceuticals, biopharmaceuticals and cosmeceutical) to a patient, said method comprising:
producing an electromagnetic field defined by a mnemonic profile of:

[(A-B-C-D)E],[(A₁-B₁-C₁-D₁)E₁]
where,
A and A₁ respectively define the number of 400 μs time units that the electromagnetic field pulse is on for wherein each of A and A₁ is a number between 0.1 and 10
B and B₁ respectively define is the number of 400 μs time units the field is off for wherein B is a number between 0.1 and 100, while B, is a number between 0.1 and 100;
C is a number between 1 and 255, which defines the number of times the A and B combination is repeated, while C₁ is also a number between 1 and 255 and defines the number of times the A₁ and B₁ combination is repeated;
D and D₁ respectively define the number of 400 μs time units that the field is off for wherein D is a number between 0 and 255, while D₁ is a number between 0 and 255; and
E defines the number of times the A to D envelope is executed before moving onto the [(A₁-B₁-C₁-D₁)E₁] packet, while E₁ defines the number of times the A₁ to D₁ envelope is executed before moving onto the next mnemonic profile wherein E and E1 are respectively numbers between 1 and 25; and
directing the electromagnetic field at a desired treatment area in or on the patient, which area is also exposed to the therapeutic substance.

According to the method of the invention, therapeutic molecules can be effectively driven into a tissue. In particular, the method of the invention allows transfer of therapeutic molecules, such as pharmaceuticals, across a range of tissues types, such as skin, lung tissue, tracheal tissue, nasal tissue, bladder tissue, placenta, vaginal tissue, rectal tissue, stomach tissue, gastrointestinal tissue, and eye or corneal tissue.

The skin is by far the largest organ of the body. It is highly impermeable to prevent loss of water and electrolytes. It is subdivided into two main layers: the outer epidermis and the inner dermis. The epidermis is the outer layer of the skin, 50 to 100 micron thick. The dermis is the inner layer of the skin and varies from 1 to 3 mm in thickness. The goal of the method of the invention is to get the drug to this layer of the skin, where the blood capillaries are located, to allow the drug to be systemically delivered. The epidermis does not contain nerve endings or blood vessels. The main purpose of the epidermis is to generate a tough layer of dead cells on the surface of the skin, thereby protecting the body from the environment. This outermost layer of epidermis is called the stratum corneum, and the dead cells that comprise it are called corneocytes or keratinocytes.

The stratum corneum is commonly modelled or described as a brick wall. The “bricks” are the flattened, dead corneocytes. Typically, there are about 10 to 15 corneocytes stacked vertically across the stratum corneum. The corneocytes are encased in sheets of lipid bilayers. The lipid bilayer sheets are separated by about 50 nm. Typically, there are about 4 to 8 lipid bilayers between each pair of corneocytes. The lipid matrix is primarily composed of ceramides, sphingolipids, cholesterol, fatty acids, and sterols, with very little water present.

Although the stratum corneum is the thinnest layer of the skin, it is also the primary barrier to the entry of molecules or microorganisms across the skin. Most molecules pass through the stratum corneum only with great difficulty. Once the molecules have crossed the stratum corneum, diffusion across the epidermis and dermis to the blood vessels occurs rapidly. The present invention provides a means to achieve this end.

In the present invention, the application of specifically selected magnetic fields to specifically selected therapeutic molecules results in directions movement of those molecules independent of carrier, solvent or base molecules.

In accordance with yet another alternative aspect of the present invention, there is provided a method of enhancing or expanding a cellular cohort in or on a patient said method comprising:
producing an electromagnetic field defined by a mnemonic profile of:

[(A-B-C-D)E],[(A₁-B₁-C₁-D₁)E₁]
where,
A and A₁ respectively define the number of 400 μs time units that the electromagnetic field pulse is on for wherein each of A and A₁ is a number between 0.1 and 10;
B and B₁ respectively define is the number of 400 μs time units the field is off for wherein B is a number between 0.1 and 100, while B₁ is a number between 0.1 and 100;
C is a number between 1 and 255, which defines the number of times the A and B combination is repeated, while C₁ is also a number between 1 and 255 and defines the number of times the A₁ and B₁ combination is repeated;
D and D₁ respectively define the number of 400 μs time units that the field is off for wherein D is a number between 0 and 255, while D₁ is a number between 0 and 255;
E defines the number of times the A to D envelope is executed before moving onto the [(A₁-B₁-C₁-D₁)E₁] packet, while E₁ defines the number of times the A₁ to D₁ envelope is executed before moving onto the next mnemonic profile wherein E and E1 are respectively numbers between 0.1 and 25; and
directing the electromagnetic field at a desired treatment area of a patient.

### Non-Limiting Illustration of the Invention

Further features of the present invention are more fully described in the following non-limiting Examples. This description is included solely for the purposes of exemplifying the present invention. It should not be understood as a restriction on the broad description of the invention as set out above.

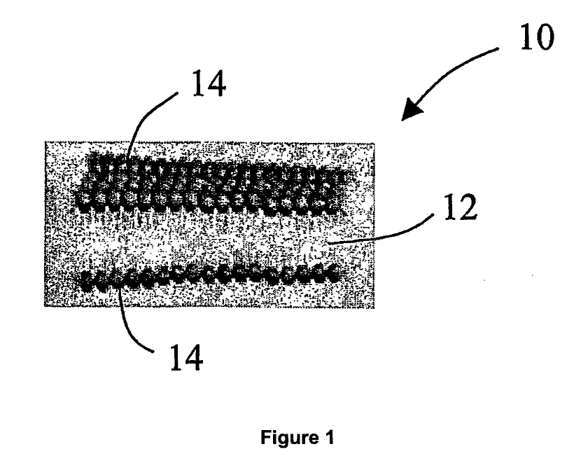

Referring to the drawings, in FIG. 1 a portion of a stratum corneum lipid bilayer structure 10 is shown diagrammatically, the lipid bilayer structure 10 having an oily core portion 12 formed of aligned hydrocarbon chains, and charged head portions 14.

During normal conditions, the bilayer structure 10 serves to prevent particles having a size greater than approximately 1 nm from passing through.

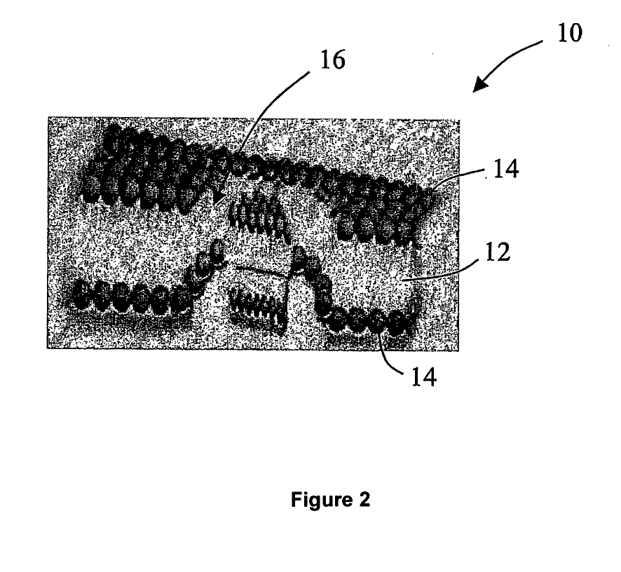

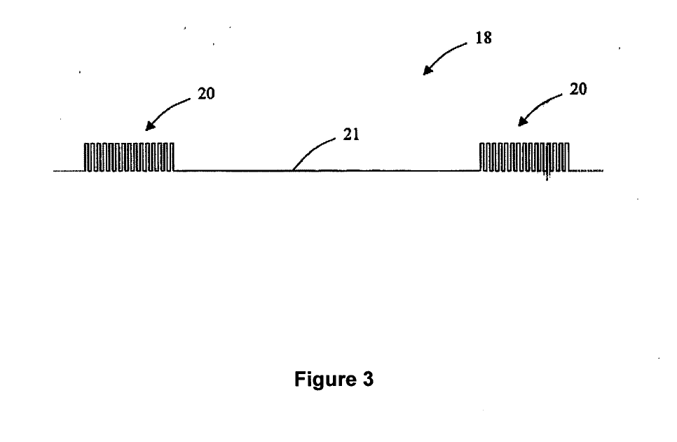

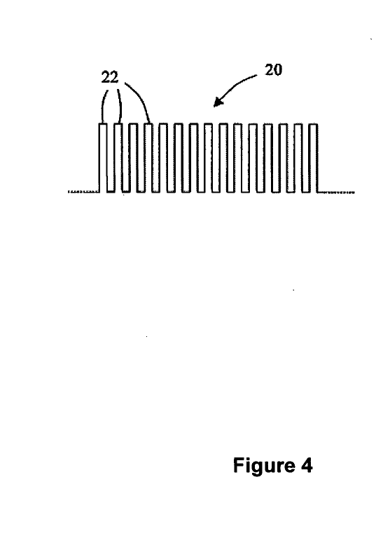

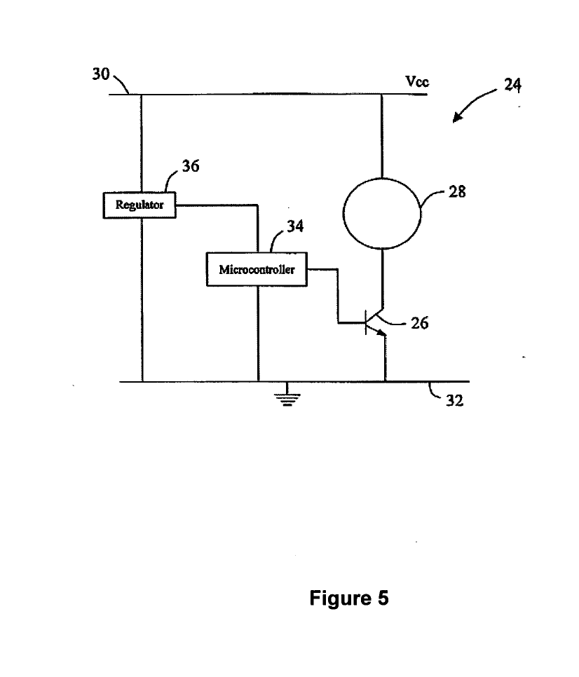

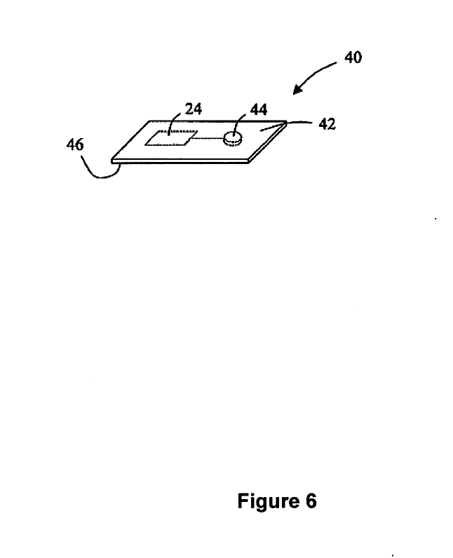

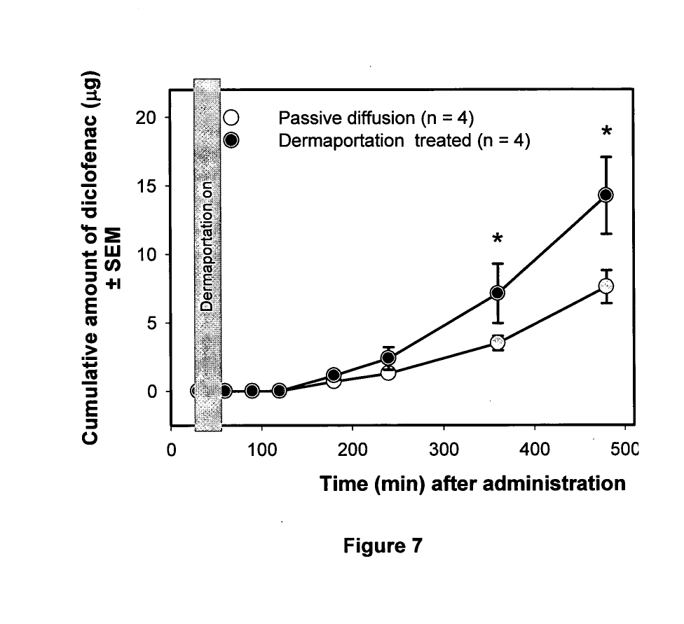

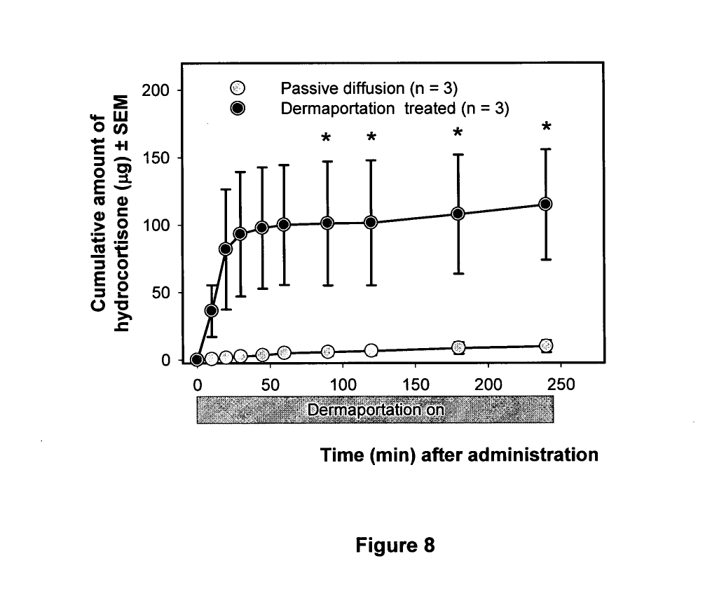

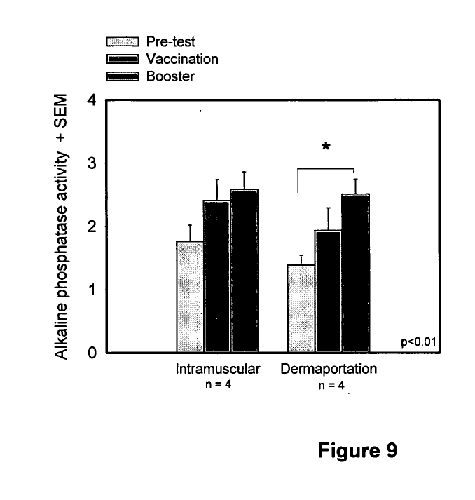

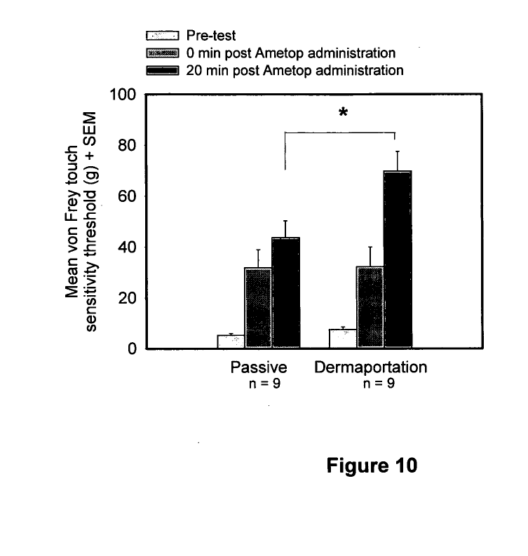

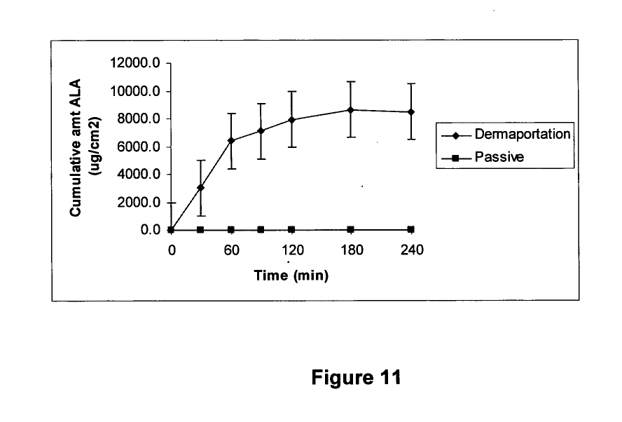

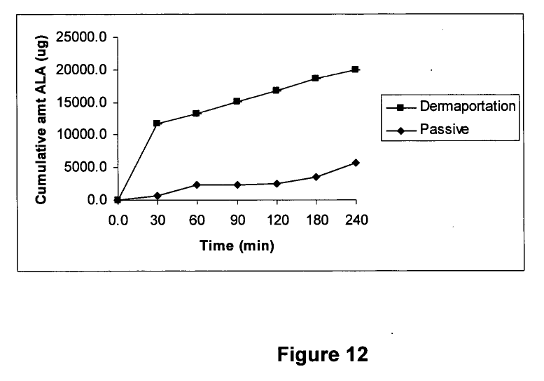

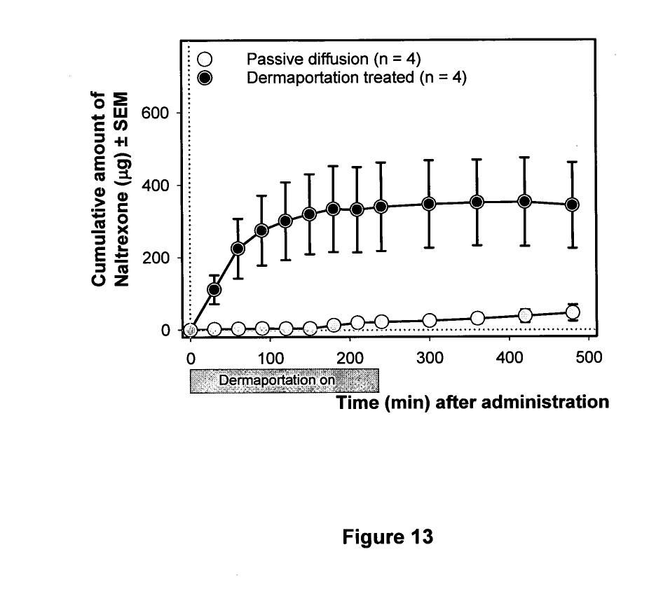

The inventor of the present invention has discovered that by applying a relatively low power electromagnetic field of particular pattern as defined above to the stratum corneum, it is possible to cause at least some of the lipids to alter their relationship with adjacent lipids in such a way as to create a pore or area of greater lipophilicity in the stratum corneum through which therapeutic substances may pass. In the present example, the electromagnetic field of predetermined pattern causes an annular aperture 16 to be defined in the stratum corneum as shown diagrammatically in FIG. 2, the annular aperture 16 being temporarily present during application of the electromagnetic field, and the structure of the stratum corneum reverting back to the barrier structure shown in FIG. 1 when the electromagnetic field is removed

The inventor of the present invention has discovered that by applying an energisation signal 18 of the general pattern shown in FIG. 3 to control circuitry of an electromagnetic field generation device such as a coil or capacitive plate the desired effect of creating a temporary aperture in the stratum corneum is achieved. The energisation signal 18 has a general pattern which comprises alternating active and inactive signal portions, the active signal portions containing a plurality of voltage pulses and the inactive signal portions containing no voltage pulses.

Energisation pulses 22 are shown more particularly in an enlarged view of the energisation signal packet 20 shown in FIG. 4. The duration of each energisation pulse 22 may be of the order of 1 μs to 1 s, more particularly 25 μs to 100 ms

In the present example, the time duration of the inactive portion 21, that is the time between successive active portions 20, is greater than the time duration of an active portion 20.

In the present example, the duration of each energisation signal pulse 22 is approximately 400 μs, the duty cycle of each of the energisation signal pulses 22 is approximately 50%, and the time duration of each inactive signal portion 21 is 15 times greater than the time duration of each active signal portion 20, although it will be understood that other variations are possible. Each energisation signal pulse 22 in the present example is of generally rectangular shape.

It will be understood that by applying energisation signal pulses 22 of generally rectangular shape to control circuitry of an electromagnetic field generation device such as a coil, active electromagnetic field portions separated by in active electromagnetic field portions are produced, with each active electromagnetic field portion containing packets of electromagnetic field pulses are produced at a spacing determined by the duration of an inactive electromagnetic field portions, and each inactive electromagnetic field portion containing no electromagnetic field pulses.

The inventor of the present invention has also discovered that the present transdermal delivery technique makes it possible to accurately target within 3 dimensions a desired treatment area by locating the electromagnetic field generation device (in this example a coil) above the desired treatment area, and modifying the packet frequency so as to influence the stratum corneum bilayers with little or no detectable effect in surrounding tissue.

Referring to FIG. 5, circuitry 24 is shown for effecting generation of an electromagnetic signal having a pattern suitable for causing an aperture to be produced in a stratum corneum.

The circuitry 24 includes a solid state switching device, in this example in the form of a bipolar transistor 26 connected in series with an electromagnetic field generation device, in this example in the form of a coil 28. Switching of the transistor 26 and thereby energisation of the coil 28 is controlled using control circuitry, in this example in the form of a microcontroller 34 preprogrammed to generate a biasing signal on the base of the transistor 26 corresponding in general pattern to the energisation signal 18 shown in FIG. 3. However, it will be understood that other arrangements for effecting controlled switching of the transistor 26 are envisaged.

In this example, a voltage regulator 36 is also provided to produce a regulated voltage necessary for the microcontroller 34, although it will be understood that for microcontrollers or other control circuitry which do not require a regulated voltage supply, the regulator 36 may be omitted.

As shown in FIG. 6, an apparatus 40 for facilitating transdermal delivery of therapeutic substances may take the form of a generally flat rectangular member which for example may be formed of plastics material. The apparatus 40 includes a body portion 42 having embedded circuitry 24 and a battery 44 for supplying power to the circuitry 24.

During use, the apparatus 40 may be placed adjacent a portion of the skin through which it is desired to introduce therapeutic substances and the circuitry 24 activated so as to cause opening of an aperture in the stratum corneum adjacent the apparatus 40.

The therapeutic substance may be disposed on a surface 46 of the body portion 42, may be applied directly to the skin, or may be introduced on to the skin in any other suitable way.

It will be appreciated that the amount of energy required to carry out the present transdermal delivery technique is approximately 1000 times less than the corresponding energy levels required for iontophoresis and electroporation transdermal delivery techniques. As a consequence, the present technique is ideally suited to implementation in compact, portable and disposable applications, in particular for outpatient and homecare use.

### Dermaportation Studies

The following table summarises the field parameters employed in the following studies undertaken with different fields and drugs in in vitro Franz-type diffusion models.

| Field parameters | Compound | Skin model |
|---|---|---|
| [(1, 1, 12, 255)5], [(1, 35, 72, 0)2] | Diclofenac diethylammonium salt | Human stomach epidermis |
| [(1, 1, 12, 255)5], [(1, 35, 72, 0)2] | Hydrocortisone in PBS | Human stomach epidermis |
| [(1, 1, 12, 255)5], [(1, 35, 72, 0)2] | GLANVAC vaccine | In vivo, sheep |
| [(1, 1, 24, 255)5], [(1, 35, 72, 0)2] | Tetracaine HCL | Land race piglet Ear Epidermis |
| [(1, 1, 12, 255)5], [(1, 35, 72, 0)2] | Tetracaine (4%) | Land race piglet ear epidermis |
| [(1, 1, 12, 255)5], [(1, 35, 72, 0)2] | Ametop (tetracaine gel) | In vivo, human |
| [(1, 1, 12, 255)5], [(1, 35, 72, 0)2] | 5-aminolevulinic acid | Human stomach epidermis |
| [(1, 1, 12, 255)5], [(1, 35, 72, 0)2] | Naltrexone in PBS | Human stomach epidermis |
| [(1, 1, 12, 255)5], [(1, 35, 72, 0)2] | 5 mg/ml Lidocaine HCL in PBS | Adult pig stomach epidermis |
| [(1, 1, 12, 255)5], [(1, 35, 72, 0)2] | 5 mg/ml Lidocaine HCL in PBS | Piglet ear epidermis, mixed pig |
| | | strains |
| [(1, 1, 12, 255)5], [(1, 35, 72, 0)2] | Lidocaine in PBS | Mixed breed, piglet ear epidermis |
| [(1, 1, 12, 255)5], [(1, 35, 72, 0)2] | Lidocaine HCL, prilocaine HCL | Adult pig belly epidermis |
| | combination,, PBS | (unknown breed) |
| [(1, 1, 12, 255)5], [(1, 35, 72, 0)2] | Lidocaine in PBS | Adult pig stomach epidermis |
| [(1, 1, 255, 0)1], [(1, 1, 255, 0)1] | | |
| [(1, 1, 12, 255)5], [(1, 35, 72, 0)2] | 5 mg/ml Lidocaine HCL in PBS | Human stomach epidermis (1 |
| [(1, 1, 12, 255)5], [1, 35, 72, 0)1] | | donor) |
| [(1, 1, 12, 255)5], [(1, 35, 72, 0)2] | 5 mg/ml Lidocaine HCL in PBS | Piglet stomach epidermis |
| [(1, 1, 12, 255)10], [(1, 35, 72, 0)2] | | |
| [(1, 1, 12, 255)5], [(1, 35, 72, 0)2] | 5 mg/ml Lidocaine HCL in PBS | Piglet ear epidermis |
| [(1, 1, 24, 255)5], [(1, 35, 72, 0)2] | | |
| [(1, 1, 12, 255)5], [(1, 35, 72, 0)2] | Lidocaine HCL | Mixed breed, piglet ear epidermis |
| [(1, 1, 24, 255)5], [(1, 35, 72, 0)2] | | |
| [(1, 1, 24, 255)5], [(1, 35, 72, 0)2] | Lidocaine Hcl 5 mg/ml | Land race piglet Ear Epidermis |
| [(1, 1, 12, 255)5], [(1, 35, 72, 0)2] | Lidocaine HCL 5 mg/ml | Land race piglet ear epidermis |
| [(1, 1, 2, 255)5], [(1, 1, 2, 255)5] | | |
| [(1, 1, 12, 255)5], [(1, 35, 72, 0)2] | Lidocaine HCL 5 mg/ml | Land race piglet ear epidermis |
| [(2.5, 255, 3), 255)10], [(2.5, 255, 3), | | |
| 255)10] | | |
| [(1, 1, 12, 255)5], [(1, 35, 72, 0)2] | Lidocaine HCL 5 mg/ml | Land race piglet ear epidermis |
| [(2.5, 255, 3), 255)10], [(2.5, 255, 3), | | |
| 255)10] | | |
| [(1, 1, 12, 255)5], [(1, 35, 72, 0)2] | Lidocaine HCL 5 mg/ml | Land race piglet ear epidermis |
| [(2.5, 2.5, 2)25000)5], [(−2.5, −2.5, | | |
| 2)25000)1] | | |
| [(1, 1, 12, 255)5], [(1, 35, 72, 0)2] | Lidocaine HCL 5 mg/ml | Land race piglet ear epidermis |
| [(2.5, 2.5, 2)25000)5], [(−2.5, −2.5, | | |
| 2)25000)1] | | |

A. Dermaportation Enhanced Skin Diffusion of a Commercially Available Topical Diclofenac Formulation

Human epidermis was mounted in vertical Franz type diffusion cells (stratum corneum facing up), according to the method of Kligman and Christophers (1963). Voltaren Emulgel® (1 g containing 1.16% diclofenac diethylammonium salt; Novartis) was applied to the donor compartment of diffusion cells, with PBS in the receptor compartment (3.0 mL; stirred continuously; 37° C.). Dermaportation coils were placed around the exterior of 4 cells, with 4 additional cells without coils (passive control). Dermaportation energy was applied from time 30-60 min. Samples of the receptor solution were removed and replaced with fresh buffer over an 8 h period. All samples were analysed for diclofenac content by HPLC with UV detection by a validated method. The cumulative amount of diclofenac in receptor versus time was plotted and flux values calculated from the slopes per group. Statistical analyses used a t-test on the delta scores from the zero time point to the time point in question.

Dermaportation enhanced skin penetration of diclofenac (flux 2.97 μg/h) compared with passive administration (flux 1.58 μg/h). At 8 h the cumulative amount penetrated was 14.25 μg for Dermaportation and 7.59 μg for passive diffusion. The t-test delta score evaluation showed that at 360 min and 480 min Dermaportation cells had reached a far higher concentration than at the previous time points. Passive diffusion did not reach diffusion levels different from 0 at any time point. This demonstrates that Dermaportation outperformed passive diffusion (FIG. 7).

B. Dermaportation Increases Hydrocortisone Penetration of Human Epidermis Ex Vivo

Human skin was obtained following abdominoplasty surgery under existing approval from the Human Research Ethics Committee of Curtin University.

The epidermis was heat separated from the dermis using standard procedures (Kligman and Christophers 1963). The epidermis was mounted in Franz-type diffusion cells with the stratum corneum facing the donor compartment. Skin integrity was determined by conductance measurement. The receptor compartment was filled with 20:80 ethanol:phosphate buffered saline pH 7.4, stirred continuously and maintained at 37° C. throughout the experiment. Hydrocortisone solution (1 mL of saturated solution in 20:80 ethanol:phosphate buffered saline pH 7.4; solubility approx 1 mg/mL) was applied to the donor side of the epidermis.

Dermaportation was applied from time 0 to 4 h. Samples were removed from the receptor fluid at time points up to 4 h. At each time point the receptor fluid volume was replaced with fresh receptor solution preheated to 37° C. Four Dermaportation cells and three passive cells (no Dermaportation) were conducted using skin from the abdominal region of a female donor. The content of hydrocortisone in receptor fluid samples was analysed by HPLC with ultraviolet detection using a validated assay procedure.

The cumulative amount of hydrocortisone in receptor versus time was plotted and flux values calculated from the slopes per group.

Dermaportation enhanced the skin penetration of hydrocortisone (flux 2.44 μg/cm²/h), when compared that of passive administration (flux 0.08 μg/cm²/h). At 30 min the cumulative amount penetrated was 93.22 μg for Dermaportation and 2.51 μg for passive diffusion. The largest increase in Dermaportation-related diffusion was achieved in the first 40 min (see FIG. 8).

C. Dermaportation Delivered Sheep Vaccination

Subjects: 8 Merino whethers (desexed male sheep, 12 months) were randomly assigned to Dermaportation enhanced topical vaccination, or intramuscular vaccination. Animal care was according to protocols and guidelines approved by the European Communities Council Directive of 24 Nov. 1986 (86/609/EEC).

Vaccination: Four days before the first treatment, a section of wool (10 cm×10 cm) was shorn for the topical vaccination. The fleece was left intact. The sheep assigned to the Dermaportation treatment had the Dermaportation device strapped on the clipped areas on their backs. Through a hole in the middle of the Dermaportation coil, 2 mL GLANVAC™ was poured, and the Dermaportation system was activated for 30 min. The control group received an intramuscular injection of 2 mL GLANVAC™.

Samples: Ten (10) mL blood samples were collected (jugular vein; into vacutainer with sodium citrate) at 1 week before vaccination, 2 weeks after, and 2 weeks post booster vaccination. The period between vaccination and booster was 4 weeks. Samples were centrifuged and the plasma frozen (−20° C.) for subsequent analysis to establish background antibody levels. The extent of anti-body production was measured using a colorimetric immuno-assay. GIANVAC™ was pipetted into wells of a Nunc maxi-sorb plate, which was subsequently rinsed and then the plasma added. The proteins were allowed to adsorb for 10 min, and then the wells were rinsed. Subsequently, a solution of rabbit anti-sheep IgG conjugated with alkaline phosphatase was pipetted into the wells (10 min). The activity of the alkaline phosphatase was measured using t p-nitrophenyl phosphatase. The alkaline phosphatase activity was measured at 450 nm absorbance and analyzed statistically.

The Dermaportation treated group had a similar immune response (total IgG) to the intramuscular group (FIG. 9). The within subjects delta scores showed that the Dermaportation treated group had a significant increase in immune response due to the two topical treatments (p<0.01).

D. Effect of Dermaportation on Time of Onset of Anaesthesia after Tetracaine Gel in Healthy Human Volunteers

Healthy adult volunteers (5 females, 2 males) participated in the study (ethical approval granted by the HREC of Curtin University). The study was carried out under the Guidelines of the National Health and Medical Research Council of Australia.

A 200 mg aliquot of Ametop® gel (4% tetracaine; Smith and Nephew) was applied to each of four arm sites, 2 per each upper arm. Two active and two passive Dermaportation coils (OBJ Limited, Perth, Australia) randomised over the arms. The distribution was double-blind. Dermaportation was applied for 20 min. The coils were then removed, the sites wiped clean with tissue paper and functional testing initiated.

Touch sensitivity was measured with an electronic von Frey device (Somedic). Three testing sessions were conducted, i.e. at 10 min before Ametop administration (pre-test), at 0, and 20 min post Ametop® administration. The volunteers were instructed to close their eyes during the trials.

The von Frey probe was held perpendicular to the upper arm, and gently pushed against the skin at each administration site. The subjects were instructed to verbally acknowledge whenever they noticed the touch with the von Frey probe. The force measured at the touch detection point was registered electronically. For each application site, three successive trials per session were conducted and averaged.

The touch sensitivity before the start of the administration of tetracaine was similar for the passive and active treatment sites. Twenty minutes after the end of the Ametop® administration, Dermaportation treated sites were less sensitive to the mechanical von Frey stimulation than the passively treated sites (Frey 20 min post: F_{1,16}=5.85; p<0.05). FIG. 10 shows that the touch threshold increases with each session, demonstrating successful anaesthesia.

E. Influence of Dermaportation on Human Epidermal Penetration of the 5-Aminolevulinic Acid

Overview: ALA solution was applied to the surface of human epidermis for a contact period of up to 4 h. Dermaportation applied continuously from 0 to 4 h was compared to passive diffusion. ALA penetrating the epidermis would diffuse into a receptor fluid of phosphate buffered saline (pH 7.4: PBS). The receptor fluid was analysed for ALA content by high performance liquid chromatography (HPLC).

In vitro epidermal penetration: Human skin was obtained following abdominoplasty surgery under existing approval from the Human Research Ethics Committee of Curtin University. Skin from a female donor was used in this study. The epidermis was heat separated from the dermis using standard procedures (Kligman and Christophers 1963). The epidermis was mounted in Franz-type diffusion cells with the stratum corneum facing the donor compartment. Skin integrity was determined by conductance measurement.

The receptor compartment was filled with phosphate buffered saline pH 7.4, stirred continuously and maintained at 37° C. throughout the experiment. ALA solution (2% w/v of ALA in phosphate buffered saline pH 7.4) was applied to the donor side of the epidermis. Dermaportation (cycle 2) was applied from time 0 to 4 h. Samples were removed from the receptor fluid at time 0, 30, 60, 90, 120, 180, 240 min. At each time point the receptor fluid volume was replaced with fresh phosphate buffered saline preheated to 37° C. Four Dermaportation cells and three passive cells (no Dermaportation) were conducted using skin from the abdominal region of a female donor. The content of ALA in receptor fluid samples was analysed by HPLC with ultraviolet detection using a validated assay procedure. The experiment was then repeated with a donor solution containing 20% ALA in phosphate buffered saline, with and without Dermaportation cycle 2 applied for 4 h (n=2).

The data is presented in FIGS. 11 and 12—At 4 h approximately 40% of the applied dose of ALA (with Dermaportation only) had penetrated to the receptor. It is likely that depletion of the drug in the donor occurred between 60-120 min, therefore limiting the diffusion rate after that time. Consequently a follow-up experiment was conducted with a donor concentration of 20% ALA (as below).

F. Dermaportation Increases Naltrexone Penetration of Human Epidermis Ex-Vivo

Human skin was obtained following abdominoplasty surgery under existing approval from the Human Research Ethics Committee of Curtin University.

The epidermis was heat separated from the dermis using standard procedures (Kligman and Christophers 1963). The epidermis was mounted in Franz-type diffusion cells with the stratum corneum facing the donor compartment. Skin integrity was determined by conductance measurement. The receptor compartment was filled with phosphate buffered saline pH 7.4, stirred continuously and maintained at 37° C. throughout the experiment. Naltrexone (0.5% w/v) in phosphate buffered saline (pH 7.4) was applied to the donor side of the epidermis.

Dermaportation was applied from time 0 to 4 h. Samples were removed from the receptor fluid at time points up to 8 h. At each time point the receptor fluid volume was replaced with fresh receptor solution preheated to 37° C. Four Dermaportation cells and 4 passive cells (no Dermaportation) were conducted using skin from the abdominal region of a female donor. The content of naltrexone in receptor fluid samples was analysed by HPLC with ultraviolet detection using a validated assay procedure.

The cumulative amount of naltrexone in receptor versus time was plotted and flux values calculated from the slopes per group.

Dermaportation enhanced the skin penetration of naltrexone (flux₀₋₂ₕ 152.6 μg/cm²/h), when compared with that of passive administration (flux₀₋₂ₕ 1.6 μg/cm²/h). In the course of the experiment, Naltrexone diffused faster and in more quantity when treated with Dermaportation for 240 min, compared with passive administration (Time by Treat interaction: F_{12,72}=4.57; p<0.05). The largest increase in Dermaportation-related diffusion was achieved in the first 150 min (150 min: F_{1,6}=6.10; p<0.05; see FIG. 13).

Numerous variations and modifications will suggest themselves to persons skilled in the relevant technical arts, in addition to those already described, without departing from the basic inventive concepts. All such variations and modifications are to be considered within the scope of the present invention, the nature of which is to be determined form the foregoing description

1.-15. (canceled)

16. An apparatus for improving the delivery of desirable substances across a biological membrane, said apparatus comprising:
means for producing and delivering an electromagnetic field to the cellular environment wherein the field delivered is defined by a mnemonic profile of:
[(A-B-C-D)E],[(A₁-B₁-C₁-D₁)E₁]
where,
A and A₁ respectively define the number of 400 μs time units that the electromagnetic field pulse is on for wherein each of A and A₁ is a number between 0.1 and 10;
B and B₁ respectively define is the number of 400 μs time units the field is off for wherein B is a number between 0.1 and 100, while B₁ is a number between 0.1 and 100 and B is equal to or greater than A and B₁ is equal to or greater than A₁; C is a number between 1 and 255, which defines the number of times the A and B combination is repeated, while C₁ is also a number between 1 and 255 and defines the number of times the A₁ and B₁ combination is repeated and C is always equal to or greater than the sum of A and B and C₁ is always equal to or greater than the sum of A₁ and B₁;
D and D₁ respectively define the number of 400 μs time units that the field is off for wherein D is a number between 0 and 255, while D₁ is a number between 0 and 255;
E defines the number of times the A to D envelope is executed before moving onto the [(A₁-B₁-C₁-D₁)E₁] packet, while E₁ defines the number of times the A₁ to D₁ envelope is executed before moving onto the next mnemonic profile wherein E and E₁ are respectively numbers between 1 and 25, and C, D and E are never equal to each other or to A or B and C₁, D₁ and E₁ are never equal to each other or to A₁ or B₁; and
wherein during use when the electromagnetic field is incident on a patient, the cellular environment across the membrane is modified.

17. An apparatus according to claim 16 wherein the electromagnetic field producing means includes a capacitively coupled plate or coil.

18. An apparatus according to claim 16 wherein the electromagnetic field producing means comprises a solid state switching device.

19. An apparatus according to claim 18 wherein the apparatus includes a control means arranged to produce an energisation signal useable to control switching of the solid state switching device, the energisation signal including a repeating energisation signal packet, each energisation signal packet including a plurality of energisation signal pulses of generally rectangular configuration.

20. An apparatus according to claim 18 wherein the solid state switching device comprises a transistor.

21. An apparatus according to claim 16 wherein the energisation of each electromagnetic pulse is at a frequency of between 1 Hz and 100 Hz

22. An apparatus according to claim 21 wherein the energisation of each electromagnetic pulse is at a frequency of between 10 Hz and 50 Hz

23. A method of transmembrane delivery of a substance to a patient, said method comprising:
producing an electromagnetic field defined by a mnemonic profile of:
[(A-B-C-D)E],[(A₁-B₁-C₁-D₁)E₁]
where,
A and A₁ respectively define the number of 400 μs time units that the electromagnetic field pulse is on for wherein each of A and A₁ is a number between 0.1 and 10;
B and B₁ respectively define is the number of 400 μs time units the field is off for wherein B is a number between 0.1 and 100, while B₁ is a number between 0.1 and 100 and B is equal to or greater than A and B₁ is equal to or greater than A₁; C is a number between 1 and 255, which defines the number of times the A and B combination is repeated, while C₁ is also a number between 1 and 255 and defines the number of times the A₁ and B₁ combination is repeated and C is always equal to or greater than the sum of A and B and C₁ is always equal to or greater than the sum of A₁ and B₁;
D and D₁ respectively define the number of 400 μs time units that the field is off for wherein D is a number between 0 and 255, while D₁ is a number between 0 and 255;
E defines the number of times the A to D envelope is executed before moving onto the [(A₁-B₁-C₁-D₁)E₁] packet, while E₁ defines the number of times the A₁ to D₁ envelope is executed before moving onto the next mnemonic profile wherein E and E₁ are respectively numbers between 1 and 25, and C, D and E are never equal to each other or to A or B and C₁, D₁ and E₁ are never equal to each other or to A₁ or B₁; and
directing the electromagnetic field at a desired treatment area of a patient's, which area is also exposed to the therapeutic substance.

24. The method of claim 23 wherein the electromagnetic field producing means includes a capacitively coupled plate or coil.

25. The method of claim 23 wherein the electromagnetic field producing means comprises a solid state switching device.

26. The method of claim 25 wherein the apparatus includes a control means arranged to produce an energisation signal useable to control switching of the solid state switching device, the energisation signal including a repeating energisation signal packet, each energisation signal packet including a plurality of energisation signal pulses of generally rectangular configuration.

27. The method of claim 25 wherein the solid state switching device comprises a transistor.

28. The method of claim 23 wherein the energisation of each electromagnetic pulse is at a frequency of between 1 Hz and 100 Hz.

29. The method of claim 28 wherein the energisation of each electromagnetic pulse is at a frequency of between 10 Hz and 50 Hz.

30. A method of enhancing or expanding a cellular cohort in or on a patient, said method comprising:
producing an electromagnetic field defined by a mnemonic profile of:
[(A-B-C-D)E],[(A₁-B₁-C₁-D₁)E₁]
where,
A and A₁ respectively define the number of 400 μs time units that the electromagnetic field pulse is on for wherein each of A and A₁ is a number between 0.1 and 10;
B and B₁ respectively define is the number of 400 μs time units the field is off for wherein B is a number between 0.1 and 100, while B₁ is a number between 0.1 and 100 and B is equal to or greater than A and B₁ is equal to or greater than A₁; C is a number between 1 and 255, which defines the number of times the A and B combination is repeated, while C₁ is also a number between 1 and 255 and defines the number of times the A₁ and B₁ combination is repeated and C is always equal to or greater than the sum of A and B and C₁ is always equal to or greater than the sum of A₁ and B₁;
D and D₁ respectively define the number of 400 μs time units that the field is off for wherein D is a number between 0 and 255, while D₁ is a number between 0 and 255;
E defines the number of times the A to D envelope is executed before moving onto the [(A₁-B₁-C₁-D₁)E₁] packet, while E₁ defines the number of times the A₁ to D₁ envelope is executed before moving onto the next mnemonic profile wherein E and E₁ are respectively numbers between 1 and 25, and C, D and E are never equal to each other or to A or B and C₁, D₁ and E₁ are never equal to each other or to A₁ or B₁; and
directing the electromagnetic field at a desired treatment area of a patient.

31. The method of claim 30 wherein the electromagnetic field producing means includes a capacitively coupled plate or coil.

32. The method of claim 30 wherein the electromagnetic field producing means comprises a solid state switching device.

33. The method of claim 32 wherein the apparatus includes a control means arranged to produce an energisation signal useable to control switching of the solid state switching device, the energisation signal including a repeating energisation signal packet, each energisation signal packet including a plurality of energisation signal pulses of generally rectangular configuration.

34. The method of claim 32 wherein the solid state switching device comprises a transistor.

35. The method of claim 30 wherein the energisation of each electromagnetic pulse is at a frequency of between 1 Hz and 100 Hz.

36. The method of claim 35 wherein the energisation of each electromagnetic pulse is at a frequency of between 10 Hz and 50 Hz.
